(19) United States
(12) Patent Application Publication
(10) Pub. No.: US 2004/0101844 A1
(43) Pub. Date: May 27, 2004

(54) Methods and compositions for producing linearly amplified amounts of (+) strand RNA

(76) Inventors: Douglas A. Amorese, Los Altos, CA (US); Diane D. Ilsley, San Jose, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/302,675

(22) Filed: Nov. 21, 2002

(51) Int. Cl. C12Q 1/68; C12P 19/34

(52) U.S. Cl. 435/6; 435/91.2

(57) ABSTRACT

Methods for producing linearly amplified amounts of (+) strand RNA from an initial mRNA source are provided. In the subject methods, an initial mRNA source, e.g., total RNA, is converted to double-stranded cDNA using a second strand cDNA promoter-primer having a promoter sequence recognized by an RNA polymerase located at its 5′ end. The resultant double-stranded cDNA is then transcribed into (+) RNA. The subject methods find use in a variety of different applications in which the preparation of linearly amplified amounts of (+) RNA is desired. Also provided are kits for practicing the subject methods.

### TECHNICAL FIELD

The technical field of this invention is the enzymatic amplification of nucleic acids.

### BACKGROUND OF THE INVENTION

The characterization of cellular gene expression finds application in a variety of disciplines, such as in the analysis of differential expression between different tissue types, different stages of cellular growth or between normal and diseased states. Fundamental to differential expression analysis is the detection of different mRNA species in a test population, and the quantitative determination of different mRNA levels in that test population. However, the detection of rare mRNA species is often complicated by one or more of the following factors: cell heterogeneity, paucity of material, or the limits of detection of the assay method. Thus, methods that amplify heterogeneous populations of mRNA that do not introduce significant changes in the relative amounts of different mRNA species facilitate this technology.

A number of methods for the amplification of nucleic acids have been described. Such methods include the “polymerase chain reaction” (PCR) (Mullis et al., U.S. Pat. No. 4,683,195), and a number of transcription-based amplification methods (Malek et al., U.S. Pat. No. 5,130,238; Kacian and Fultz, U.S. Pat. No. 5,399,491; Burg et al., U.S. Pat. No. 5,437, 990). Each of these methods uses primer-dependent nucleic acid synthesis to generate a DNA or RNA product, which serves as a template for subsequent rounds of primer-dependent nucleic acid synthesis. Each process uses (at least) two primer sequences complementary to different strands of a desired nucleic acid sequence and results in an exponential increase in the number of copies of the target sequence. These amplification methods can provide enormous amplification (up to billion-fold). However, these methods have limitations that make them not amenable for gene expression monitoring applications. First, each process results in the specific amplification of only the sequences that are bounded by the primer binding sites. Second, exponential amplification can introduce significant changes in the relative amounts of specific target species—small differences in the yields of specific products (for example, due to differences in primer binding efficiencies or enzyme processivity) become amplified with every subsequent round of synthesis.

Amplification methods that utilize a single primer are amenable to the amplification of heterogeneous mRNA populations. The vast majority of mRNAs carry a homopolymer of 20-250 adenosine residues on their 3′ ends (the poly-A tail), and the use of poly-dT primers for cDNA synthesis is a fundamental tool of molecular biology. “Single-primer amplification” protocols have been reported (see e.g. Kacian et al., U.S. Pat. No. 5,554,516; Van Gelder et al., U.S. Pat. No. 5,716,785). The methods reported in these patents utilize a single primer containing an RNA polymerase promoter sequence and a sequence complementary to the 3′-end of the desired nucleic acid target sequence(s) (“promoter-primer”). In both methods, the promoter-primer is added under conditions where it hybridizes to the target sequence(s) and is converted to a substrate for RNA polymerase. In both methods, the substrate intermediate is recognized by RNA polymerase, which produces multiple copies of RNA complementary to the target sequence(s) (“antisense RNA”). Each method uses, or could be adapted to use, a primer containing poly-dt for amplification of heterogeneous mRNA populations.

Amplification methods that proceed linearly during the course of the amplification reaction are less likely to introduce bias in the relative levels of different mRNAs than those that proceed exponentially. As such, they offer significant advantages over exponential amplification methods in certain embodiments. A common feature of the above methods is that they produce antisense RNA from the initial mRNA source, since the RNA promoter domain is present on the first strand cDNA primer. Depending on the particular application being performed, antisense RNAs are not always ideal.

Accordingly, there is interest in the development of linear amplification protocols that can readily produce linearly amplified amounts of (+) strand RNA from initial mRNA source.

### Relevant Literature

U.S. Pat. Nos. disclosing methods of antisense RNA synthesis include: 6,132,997; 5,932,451; 5,716,785; 5,554,516; 5,545,522; 5,437,990; 5,130,238; and 5,514,545. Antisense RNA synthesis is also discussed in Phillips and Eberwine (1996), Methods: A companion to Methods in Enzymol. 10, 283; Eberwine et al. (1992), Proc., Natl., Acad. Sci. USA 89, 3010; Eberwine (1996), Biotechniques 20, 584; and Eberwine et al. (1992), Methods in Enzymol. 216, 80.

### SUMMARY OF THE INVENTION

Methods for producing linearly amplified amounts of (+) strand RNA from an initial mRNA source are provided. In the subject methods, an initial mRNA source, e.g., total RNA, is converted to double-stranded cDNA using a second strand cDNA promoter-primer having a promoter sequence recognized by an RNA polymerase located at its 5′ end, and in many embodiments a 3′ ATG codon. The resultant double-stranded cDNA is then transcribed into (+) RNA. The subject methods find use a variety of different applications in which the preparation of linearly amplified amounts of (+) RNA is desired. Also provided are kits for practicing the subject methods.

### SUMMARY OF THE INVENTION

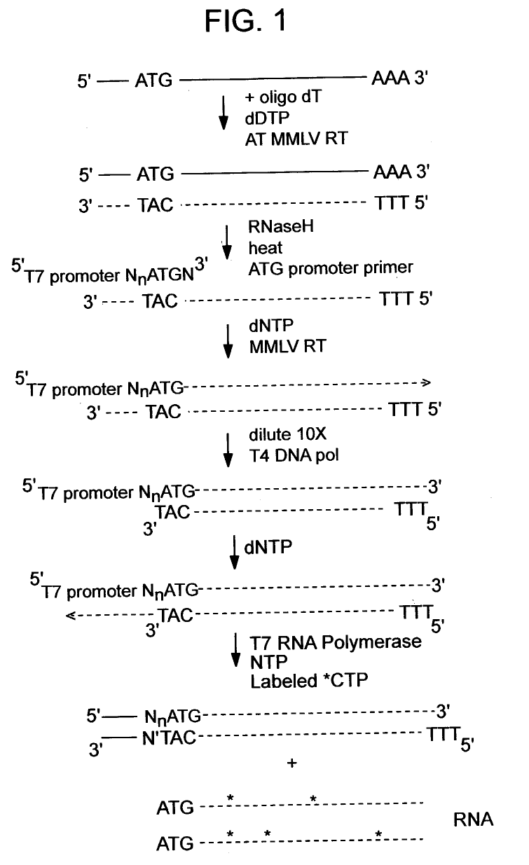

FIG. 1 provides a schematic representation of a method according to the subject invention.

### DEFINITIONS

The term “nucleic acid” as used herein means a polymer composed of nucleotides, e.g. deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

The term “functionalization” as used herein relates to modification of a solid substrate to provide a plurality of functional groups on the substrate surface. By a “functionalized surface” as used herein is meant a substrate surface that has been modified so that a plurality of functional groups are present thereon.

The term “array” encompasses the term “microarray” and refers to an ordered array presented for binding to ligands such as polymers, polynucleotides, peptide nucleic acids and the like.

The terms “reactive-site”, “reactive functional group” or “reactive group” refer to moieties on a monomer, polymer or substrate surface that may be used as the starting point in a synthetic organic process. This is contrasted to “inert” hydrophilic groups that could also be present on a substrate surface, e.g., hydrophilic sites associated with polyethylene glycol, a polyamide or the like.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure. In the practice of the instant invention, oligomers will generally comprise about 2-50 monomers, preferably about 2-20, more preferably about 3-10 monomers.

The term “ligand” as used herein refers to a moiety that is capable of covalently or otherwise chemically binding a compound of interest. The arrays of solid-supported ligands produced by the methods can be used in screening or separation processes, or the like, to bind a component of interest in a sample. The term “ligand” in the context of the invention may or may not be an “oligomer” as defined above. However, the term “ligand” as used herein may also refer to a compound that is “pre-synthesized” or obtained commercially, and then attached to the substrate.

The term “sample” as used herein relates to a material or mixture of materials, typically, although not necessarily, in fluid form, containing one or more components of interest.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

An “array,” includes any two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse-jets of either polynucleotide precursor units (such as monomers) in the case of in situ fabrication, or the previously obtained polynucleotide. Such methods are described in detail in, for example, the previously cited references, including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned these references are incorporated herein by reference. Other drop deposition methods can be used for fabrication, as previously described herein. Also, instead of drop deposition methods, photolithographic array fabrication methods may be used such as described in U.S. Pat. No. 5,599,695, U.S. Pat. No. 5,753,788, and U.S. Pat. No. 6,329,143. Interfeature areas need not be present particularly when the arrays are made by photolithographic methods as described in those patents.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is, meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

The term “stringent hybridization conditions” as used herein refers to conditions that are that are compatible to produce duplexes on an array surface between complementary binding members, i.e., between probes and complementary targets in a sample, e.g., duplexes of nucleic acid probes, such as DNA probes, and their corresponding nucleic acid targets that are present in the sample, e.g., their corresponding mRNA analytes present in the sample. An example of stringent hybridization conditions is hybridization at 60° C. or higher and 3×SSC (450 mM sodium chloride/45 mM sodium citrate). Another example of stringent hybridization conditions is incubation at 42° C. in a solution containing 30% formamide, 1M NaCl, 0.5% sodium sarcosine, 50 mM MES, pH 6.5. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

### DESCRIPTION OF THE SPECIFIC EMBODIMENTS

Methods for producing linearly amplified amounts of (+) strand RNA from an initial mRNA source are provided. In the subject methods, an initial mRNA source, e.g., total RNA, is converted to double-stranded cDNA using a second strand cDNA prmmoter-primer having a promoter sequence recognized by an RNA polymerase located at its 5′ end, and in many embodiments a 3′ ATG codon. The resultant double-stranded cDNA is then transcribed into (+) RNA. The subject methods find use a variety of different applications in which the preparation of linearly amplified amounts of (+) RNA is desired. Also provided are kits for practicing the subject methods.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the invention components that are described in the publications which might be used in connection with the presently described invention.

As summarized above, the present invention provides methods of preparing amplified amounts of (+) strand RNA from an initial mRNA source, e.g., total RNA, as well as kits for use in practicing the subject methods. In further describing the present invention, the subject methods are discussed first in greater detail, followed by a review of representative kits for use in practicing the subject methods.

### METHODS

The subject invention provides methods for linearly amplifying an initial mRNA source into (+) strand RNA. As such, the subject invention provides methods of producing amplified amounts of (+) strand RNA from an initial amount of mRNA. By amplified amounts is meant that for each initial mRNA amplified from the initial source, multiple corresponding (+) strand RNAs are produced, where the term (+) strand RNA is defined here as ribonucleic acid having a sequence corresponding to a sequence found the initial mRNA. By corresponding is meant that the; (+) strand RNA shares a substantial amount of sequence identity, if not complete sequence identity, with the sequence of the initial mRNA from which it was amplified, where substantial amount means at least 95% usually at least 98% and more usually at least 99%, where sequence identity is determined using the BLAST algorithm, as described in Altschul et al. (1990), J. Mol. Biol. 215:403-410 (using the published default setting, i.e. parameters w=4, t=17). Generally, the number of corresponding (+) strand RNA molecules produced for each initial mRNA during the subject linear amplification methods will be at least about 50, usually at least about 200, where the number may be as great as 600 or greater, but often does not exceed about 1000.

In the first step of the subject methods, an initial mRNA source or sample is subjected to a series of enzymatic reactions under conditions sufficient to ultimately produce double-stranded DNA for each initial mRNA in the sample, where the product double-stranded cDNA molecule is characterized by having an RNA polymerase promoter located at or near the 5′ terminus of the second strand cDNA molecule. As such, during this first step, an RNA polymerase promoter region is incorporated into the resultant product, which region is employed in the second step of the subject methods, i.e. the transcription step described in greater detail, below. A feature of the subject methods is that the RNA polymerase promoter region is a domain on the primer employed for second strand cDNA synthesis, as described in greater detail below.

The initial mRNA may be present in a variety of different samples, where the sample will typically be derived from a physiological source. The physiological source may be derived from a variety of eukaryotic sources, with physiological sources of interest including sources derived from single-celled organisms such as yeast and multicellular organisms, including plants and animals, particularly mammals, where the physiological sources from multicellular organisms may be derived from particular organs or tissues of the multicellular organism, or from isolated cells derived therefrom. In obtaining the sample of RNA to be analyzed from the physiological source from which it is derived, the physiological source may be subjected to a number of different processing steps, where such processing steps might include tissue homogenization, cell isolation and cytoplasm extraction, nucleic acid extraction and the like, where such processing steps are known to those of skill in the art. Methods of isolating RNA from cells, tissues, organs or whole organisms are known to those of skill in the art and are described in Maniatis et al. (1989), Molecular Cloning: A Laboratory Manual 2d Ed. (Cold Spring Harbor Press). In certain embodiments, the initial mRNA sample is a total RNA sample, i.e., a total RNA preparation, where the total RNA sample will typically be derived from a physiological source, as described above.

Depending on the nature of the primer employed during first strand synthesis, as described in greater detail below, the subject methods can be used to produce amplified amounts of (+) strand RNA corresponding to substantially all of the mRNA present in the initial sample, or to a proportion or fraction of the total number of distinct mRNAs present in the initial sample. By substantially all of the mRNA present in the sample is meant more than 90%, usually more than 95%, where that portion not amplified is solely the result of inefficiencies of the reaction or the enzyme and not intentionally excluded from amplification.

The linear amplification reaction employed in the subject methods includes a first double stranded cDNA synthesis step, which first step includes two sub-steps: (a) a first step in which first strand cDNA complementary to the initial mRNA being amplified is prepared; and (b) a second step where this resultant hybrid molecule is then converted to a double stranded cDNA molecule.

In this first substep, i.e., the first strand cDNA hybrid molecule preparation substep, a first strand cDNA primer is employed to enzymatically produce the desired first strand cDNA molecule. In many embodiments, the employed first strand cDNA primer molecule includes a poly-dt region for hybridization to the poly-A tail of the initial mRNA. The poly-dt region is sufficiently long to provide for efficient hybridization to the poly-A tail, where the region typically ranges in length from 10-50 nucleotides in length, usually 10-25 nucleotides in length, and more usually from 10 to 20 nucleotides in length.

Where one wishes to amplify only a portion of the mRNA species in the sample, one may optionally provide for a short arbitrary sequence 3′ of the poly-dT region, where the short arbitrary sequence will generally be less than 5 nucleotides in length and usually less than 4 nucleotides in length, e.g., about 3 nucleotides in length, where the dNTP immediately adjacent to the poly-dt region will not be a T residue and usually the sequence will comprise no T residue. Such short 3′ arbitrary sequences are described in Ling and Pardee (1992), Science 257, 967. In certain embodiments, the primer will be a “lock-dock” primer, in which immediately 3′ of the poly-dT region is either a “G”, “C”, or “A” such that the primer has the configuration of 3′-XTTTTTTT . . . 5′, where X is “G”, “C”, or “A”.

In the first step of the subject methods, the first strand cDNA primer is hybridized with a sufficient amount of an initial mRNA (containing the mRNA to be amplified) sample/source, e.g., total RNA (as described above) to produce primer-mRNA hybrid molecules which are then converted to first strand cDNA hybrid molecules by subjecting the primer/mRNA hybrids to primer extension reaction conditions, i.e., first strand cDNA synthesis conditions. As such, the first strand cDNA primer is contacted with the mRNA of initial mRNA source under conditions that allow the poly-dT site to hybridize to the poly-A tail present on most mRNA species in the initial mRNA sample. The resultant duplexes are then maintained under conditions sufficient to produce first strand cDNA molecules from the hybrid molecules. Specifically, the resultant duplexes are maintained in the presence of reagents necessary to, and for a period of time sufficient to, convert the primer-mRNA hybrids to first strand cDNA hybrid molecules. Depending on the particular conditions employed, the product first strand cDNA molecules may be present as single stranded molecules or as duplex structures, in which they are hybridized to the template mRNA molecules, i.e., as duplex mRNA/first strand cDNA hybrid molecules.

To produce the desired first strand cDNA from the initial primer-mRNA hybrids, the initial hybrids are typically contacted with a sufficient amount of an RNA-dependent DNA polymerase, i.e., a reverse transcriptase. Representative reverse transcriptases include, but are not limited to: Moloney murine leukemia virus (MMLV-RT), avian myeloblastosis virus (AMV-RT), bovine leukemia virus (BLV-RT), Rous sarcoma virus (RSV) and human immunodeficiency virus (HIV-RT) catalyze each of these activities. In certain embodiments, the reverse transcriptase employed is one that lacks RNaseH activity, i.e., an RNase H− reverse transcriptase. A representative example of an RNase H-reverse transcriptase that may be employed is MMLV reverse transcriptase lacking RNaseH activity (described in U.S. Pat. No. 5,405,776)(e.g. Superscript II™). The reverse transcriptase first strand cDNA from the initial primer-mRNA hybrid in the presence of additional reagents which include, but are not limited to: dNTPs; monovalent and divalent cations, e.g. KCl, MgCl₂; sulfhydryl reagents, e.g. dithiothreitol; and buffering agents, e.g. Tris-Cl. Production of the first strand cDNA from the primer-mRNA hybrid results from the extension of the hybridized promoter-primer by the RNA-dependent DNA polymerase activity of the employed reverse transcriptase. The above first substep results in the production of first strand cDNA hybrid molecules, as described above, where the molecules may either be single stranded molecules (e.g., where an RNaseH+ reverse transcriptase is employed) or duplex mRNA/first strand cDNA molecules (e.g., where an RNaseH− reverse transcriptase is employed).

The above resultant first strand cDNA molecules are then converted to double-stranded cDNA molecules in the second substep of the subject methods. A feature of this substep is that the primer employed in the second strand cDNA synthesis is a promoter primer. In other words, a second strand cDNA promoter primer is employed to enzymatically convert the product molecules of the first substep to double stranded cDNA molecules. The second strand cDNA promoter-primer employed in the subject methods includes an RNA polymerase promoter domain or region located at least proximal to the 5′ end of the primer, where the promoter domain or region is one that is in an orientation capable of directing transcription of (+) strand RNA from the resultant double stranded cDNA molecules. By at least proximal to is meant at least near or adjacent to, if not at, the 5′ terminus, where in certain representative embodiments, the 5′ most base of the promoter domain is from about 0 to about 10, often from about 0 to about 5 bases from the 5′ terminal base of the promoter primer.

A number of RNA polymerase promoters may be used for the promoter region of the first strand cDNA primer, i.e. the promoter-primer. Suitable promoter regions will be capable of initiating transcription from an operationally linked DNA sequence in the presence of ribonucleotides and an RNA polymerase under suitable conditions. The promoter will be linked in an orientation to permit transcription of sense RNA. A linker oligonucleotide between the promoter and the DNA may be present, and if, present, will typically comprise between about 5 and 20 bases, but may be smaller or larger as desired. The promoter region will usually comprise between about 15 and 250 nucleotides, preferably between about 17 and 60 nucleotides, from a naturally occurring RNA polymerase promoter or a consensus promoter region, as described in Alberts et al. (1989) in Molecular Biology of the Cell, 2d Ed. (Garland Publishing, Inc.). In general, prokaryotic promoters are preferred over eukaryotic promoters, and phage or virus promoters most preferred. As used herein, the term “operably linked” refers to a functional linkage between the affecting sequence (typically a promoter) and the controlled sequence (the mRNA binding site). The promoter regions that find use are regions where RNA polymerase binds tightly to the DNA and contain the start site and signal for RNA synthesis to begin. A wide variety of promoters are known and many are very well characterized. Representative promoter regions of particular interest include T7, T3 and SP6 as described in Chamberlin and Ryan, The Enzymes (ed. P. Boyer, Academic Press, New York) (1982) pp 87-108.

In certain embodiments, the second strand cDNA promoter primer is further characterized in that it includes an ATG codon at or near, i.e., at least proximal to, its 3′ terminus. By at least proximal to is meant at least near or adjacent to, if not at, the 3′ terminus, where in certain representative embodiments, the 3′ most base of ATG codon is from about 0 to about 10, often from about 0 to about 5 bases from the 3′ terminal base of the promoter primer.

In certain embodiments, the second strand cDNA primer further includes a spacer domain 3′ of the RNA polymerase promoter domain, where the spacer domain may be made up of one or more nucelotide residues, of any base, e.g., degenerate bases, universal bases, etc. In certain embodiments, the spacer domain is made up of from about 1 to 10 nt, usually from about 2 to 8 nt, including 3, 4, 5, or 6 nt, etc. In certain embodiments, the spacer is a random oligomer, e.g., hexamer, where all possible variations of this random oligomer are represented in a primer mix of second strand cDNA primers. For example, in certain embodiments where the spacer is denoted NNNNNN, this representation is intended to indicate that A, G, C, or T can appear at any position, and therefore the spacer six nucleotides of the primers in the set represent all 4096 (4⁶) possible hexamers. In those embodiments that include a 3′ ATG codon, the spacer domain is positioned between the 5′ promoter primer domain and the 3′ ATG codon. In certain embodiments, the second strand cDNA promoter primer is described by the formula:

5′-RNA polymerase promoter domain-(N)ₙ-ATG-(N)ₘ-3′

or

wherein:

N is any deoxyribonucleotide residue, e.g., A, G, C, T;

n is from about 1 to about 10, e.g., from 1 to 8, from 2 to 7, etc; and

m is 0 or an integer from about 1 to about 10, e.g., from 1 to 8, from 2 to 7, etc.

The above promoter primer is contacted with the mRNA/first strand cDNA hybrids under conditions sufficient to produce double stranded cDNAs from the initial first strand cDNAs. As such, the above promoter primers are contacted with the first strand cDNAs in the presence of a sufficient DNA polymerase under primer extension conditions sufficient to produce the desired double stranded cDNA molecules. DNA polymerases of interest include, but are not limited to, polymerases derived from E. coli, thermophilic bacteria, archaebacteria, phage, yeasts, Neurosporas, Drosophilas, primates and rodents, Reverse Transcriptases and the like. The DNA polymerase converts the initial first strand cDNAs to double stranded cDNA molecules in the presence of additional reagents which include, but are not limited to: dNTPs; monovalent and divalent cations, e.g. KCl, MgCl₂; sulfhydryl reagents, e.g. dithiothreitol; and buffering agents, e.g. Tris-Cl.

The above described second strand cDNA synthesis substep results in the production of a double-stranded cDNA molecule that includes a single stranded RNA polymerase promoter region located at the 5′ end of the second strand cDNA strand. As such, the second strand cDNA includes not only a sequence of nucleotide residues that includes a DNA copy of the mRNA template, but also additional sequences at its 5′ end that are the promoter primer employed in its synthesis. This single stranded region is then converted to a double stranded region, e.g., with use of a third polymers and dNTPs, to produced a fully double stranded structured. The 5′ promoter region of the second strand cDNA strand serves as a recognition site and transcription initiation site for an RNA polymerase in the production of (+) RNA from the double stranded cDNA molecule, which uses the first strand cDNA as a template for multiple rounds of (+) strand RNA synthesis during the next stage of the subject methods.

The primers described above and throughout this specification, e.g., the first and second strand cDNA primers, may be prepared using any suitable method, such as, for example, the known phosphotriester and phosphite triester methods, or automated embodiments thereof. In one such automated embodiment, dialkyl phosphoramidites are used as starting materials and may be synthesized as described by Beaucage et al. (1981), Tetrahedron Letters 22, 1859. One method for synthesizing oligonucleotides on a modified solid support is described in U.S. Pat. No. 4,458,066. It is also possible to use a primer that has been isolated from a biological source (such as a restriction endonuclease digest). The primers herein are selected to be “substantially” complementary to each specific sequence to be amplified, i.e.; the primers should be sufficiently complementary to hybridize to their respective targets. Therefore, the primer sequence need not reflect the exact sequence of the target, and can, in fact be “degenerate.” Non-complementary bases or longer sequences can be interspersed into the primer, provided that the primer sequence has sufficient complementarity with the sequence of the target to be amplified to permit hybridization and extension.

The next step of the subject method is the preparation of (+) strand RNA from the double-stranded cDNA prepared in the first step. During this step, the double-stranded cDNA produced in the first step is transcribed by RNA polymerase to yield (+) RNA, which shares sequence identity to the initial mRNA target from which it is amplified.

Depending on the particular protocol employed, the subject methods may or may not include a step in which the double-stranded cDNAs produced as described above are physically separated from the reverse transcriptase employed in the cDNA production step prior to the transcription step. As such, in certain embodiments, the cDNAs produced in the first step of the subject methods are separated from the reverse transcriptase employed in this first step prior to the second transcription step described in greater detail below. In these embodiments, any convenient separation protocol may be employed, including the phenol/chloroform extraction and ethanol precipitation (or dialysis), protocol as described in U.S. Pat. No. 5,554,516 and U.S. Pat. No. 5,716,785, the disclosures of which are herein incorporated by reference.

In yet other embodiments, the subject methods do not involve a step in which the double-stranded cDNA is physically separated from the reverse transcriptase following double-stranded cDNA preparation. In these embodiments, the reverse transcriptase that is present during the transcription step is rendered inactive. Thus, the transcription step is carried out in the presence of a reverse transcriptase that is unable to catalyze RNA-dependent DNA polymerase activity, at least for the duration of the transcription step. As a result, the (+) RNA products of the transcription reaction cannot serve as substrates for additional rounds of amplification, and the amplification process cannot proceed exponentially.

The reverse transcriptase present during the transcription step may be rendered inactive using any convenient protocol, including those described in U.S. Pat. No. 6,132,997; the disclosure of which is herein incorporated by reference. As described in this reference, the transcriptase may be irreversibly or reversibly rendered inactive. Where the transcriptase is reversibly rendered inactive, the transcriptase is physically or chemically altered so as to no longer able to catalyze RNA-dependent DNA polymerase activity. The transcriptase may be irreversibly inactivated by any convenient means. Thus, the reverse transcriptase may be heat inactivated, in which the reaction mixture is subjected to heating to a temperature sufficient to inactivate the reverse transcriptase prior to commencement of the transcription step. In these embodiments, the temperature of the reaction mixture and therefore the reverse transcriptase present therein is typically raised to 55° C. to 70° C. for 5 to 60 minutes, usually to about 65° C. for 15 to 20 minutes. Alternatively, reverse transcriptase may irreversibly inactivated by introducing a reagent into the reaction mixture that chemically alters the is protein so that it no longer has RNA-dependent DNA polymerase activity. In yet other embodiments, the reverse transcriptase is reversibly inactivated. In these embodiments, the transcription may be carried out in the presence of an inhibitor of RNA-dependent DNA polymerase activity. Any convenient reverse transcriptase inhibitor may be employed which is capable of inhibiting RNA-dependent DNA polymerase activity a sufficient amount to provide for linear amplification. However, these inhibitors should not adversely affect RNA polymerase activity. Reverse transcriptase inhibitors of interest include ddNTPs, such as ddATP, ddCTP, ddGTP or ddTTP, or a combination thereof, the total concentration of the inhibitor typically ranges from about 50 μM to 200 μM.

Regardless of whether the cDNA is separated from the reverse transcriptase prior to the transcription step, for the transcription step, the presence of the RNA polymerase promoter region on the double-stranded cDNA is exploited for the production of (+) strand RNA. To synthesize the (+) strand RNA, the double-stranded DNA is contacted with the appropriate RNA polymerase in the presence of the four ribonucleotides, under conditions sufficient for RNA transcription to occur, where the particular polymerase employed will be chosen based on the promoter region present in the double-stranded DNA, e.g. T7 RNA polymerase, T3 or SP6 RNA polymerases, E. coli RNA polymerase, and the like. Suitable conditions for RNA transcription using RNA polymerases are known in the art, see e.g. Milligan and Uhlenbeck (1989), Methods in Enzymol. 180, 51.

The above protocol results in the production of (+) strand RNA from an initial mRNA source. A representative protocol is shown in FIG. 1.

### UTILITY

The resultant (+) strand RNA produced by the subject methods finds use in a variety of applications. For example, the resultant (+) strand RNA can be used in expression profiling analysis on such platforms as DNA microarrays, for construction of “driver” for subtractive hybridization assays, for cDNA library construction, and the like.

Depending on the particular intended use of the subject (+) strand RNA, the (+) strand RNA may be labeled. One way of labeling which may find use in the subject invention is isotopic labeling, in which one or more of the nucleotides is labeled with a radioactive label, such as ³²S, ³²P, ³H, or the like. Another means of labeling is fluorescent labeling in which fluorescently tagged nucleotides, e.g. CTP, are incorporated into the antisense RNA product during the transcription step. Fluorescent moieties which may be used to tag nucleotides for producing labeled antisense RNA include: fluorescein, the cyanine dyes, such as Cy3, Cy5, Alexa 555, Bodipy 630/650, and the like. Other labels may also be employed as are known in the art.

In certain embodiments, the (+) strand RNA produced by the subject methods is employed as template in the preparation of labeled deoxyribonucleic acid molecules, e.g., labeled target DNA molecules. To prepare labeled target DNA molecules from the (+) strand RNA product of the subject methods, the (+) strand RNA target is typically contacted with a suitable primer, catalytic activities and other reagents required to generate labeled target nucleic acid from the (+) strand RNA template molecules. The primers may be any of a number of different kinds of primers known to those of skill in the art, including a random hexamer primers, gene specific primers, etc. The catalytic activities employed typically include an RNA-dependent DNA polymerase activity, i.e., a reverse transcriptase, which may or may not have RNase H activity, where representative reverse transcriptases are discussed above. In such, methods, the (+) strand RNA templates are contacted with the reverse transcriptase and other reagents, where the additional reagents may include, but are not limited to: dNTPs; labeled dNTPs, monovalent and divalent cations, e.g. KCl, MgCl₂; sulfhydryl reagents, e.g. dithiothreitol; and buffering agents, e.g. Tris-Cl; under conditions sufficient to produce the desired labeled target deoxyribonucleic acids, where such conditions are well known to those of skill in the art.

One broad type of application in which the subject methods of (+) strand RNA synthesis find use is nucleic acid analyte detection applications, where the subject methods are employed to generate a labeled nucleic acid analyte from an initial nucleic acid sample or source. Specific analyte detection applications of interest include hybridization assays in which the nucleic acids produced by the subject methods are hybridized to arrays of probe nucleic acids.

An “array”, unless a contrary intention appears; includes any one-, two- or three-dimensional arrangement of addressable regions bearing a particular chemical moiety or moieties (for example, biopolymers such as polynucleotide sequences) associated with that region. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

In these assays, a sample of labeled target nucleic acids, e.g., labeled (+) strand RNA or labeled target deoxyribonucleic acids (as described above) is first prepared according to the methods described above, where preparation may include labeling of the target nucleic acids with a label, e.g. a member of signal producing system. Following sample preparation, the sample is contacted with an array under hybridization conditions, whereby complexes are formed between target nucleic acids that are complementary to probe sequences attached to the array surface. The presence of hybridized complexes is then detected. Specific hybridization assays of interest which may be practiced include: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270;.5,525,464; 5,547,839; 5,580,732; 5,661,028; 5,800,992; the disclosures of which are herein incorporated by reference.

As such, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER scanner available from Agilent Technologies, Palo Alto, Calif. Other suitable apparatus and methods are described in U.S. patent applications: Ser. No. 09/846,125 “Reading Multi-Featured Arrays” by Dorsel et al.; and Ser. No. 09/430,214 “Interrogating Multi-Featured Arrays” by Dorsel et al., where these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere).

Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

### KITS

Also provided are kits for use in the subject invention, where such kits may comprise containers, each with one or more of the various reagents (typically in concentrated form) utilized in the methods, including, for example, buffers, the appropriate nucleotide triphosphates (e.g. dATP, dCTP, dGTP, dTTP, ATP, CTP, GTP and UTP), reverse transcriptase, RNA polymerase, DNA polymerase, and the second strand promoter-primer of the present invention, as well as the first strand primer. Also present in the kits may be total RNA isolation reagents, e.g., RNA extraction buffer, proteinase digestion buffer; proteinase K, etc. Also present in the kits may be one or more detergents.

Finally, the kits may further include instructions for using the kit components in the subject methods. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

The following examples are offered by way of illustration and not by way of limitation.

### EXPERIMENTAL

Total RNA extracted from HeLa cells and Spleen tissue isolated using traditional methods (eg Trizol, Qiagen) is concentrated to a final concentration of >0.3 mg/ml in a Speed-Vac.

Two labeling reactions are carried out as described below, the HeLa sample to be ultimately labeled with Cyanine 3, the spleen sample to be ultimately labeled with Cyanine 5. A solution containing 6 μg of total RNA is transferred to a microfuge tube containing 100 pMoles oligo dT primer, the solution is heated to 95° C. for 3-5 minutes and allowed to cool to room temperature. After 10 minutes at room temperature components are added to achieve final reaction conditions; 500 μM dNTP (dATP/dTTP/dGTP/dCTP), 1×MMLV reaction buffer (50 mM Tris-HCl, pH 8.3, 75 mM KCl, 3 mM MgCl₂, 10 mM DTT) and 400μ MMLV-RT. The reaction is transferred to 42° C. water bath and allowed to proceed for 60 minutes. After 60 minutes, 10 units of Rnase H are added and the incubation allowed to proceed at room temperature for 15 minutes. The reaction vials are transferred to a 95° C. waterbath for 5 minutes, 100 pMoles of ATG-Promoter primer are added to the vials and the vials are returned to the 95° C. bath for an additional 5 minutes. The solution is allowed to cool to room temperature, 200 units of MMLV-RT are added and the reaction is returned to the 42° C. incubator for 60 minutes. The reactions are transferred to a 12° C. incubator and 1-2 U T4 DNA polymerase are added to the reactions. The incubation is allowed to proceed for 15 minutes. The polymerase is denatured by incubation at 95° C. for 5 minutes.

After cooling, NTPs, Cyanine labeled CTP, reaction buffer and T7 RNA polymerase are added to the reactions and they are incubated at 37° C. for 60 minutes. Alternatively, transcription reactions are allowed to proceed in the absence of labeled nucleotides and the transcripts are labeled via random primer labeling in a separate reaction. Thus allowing either strand to be labeled; + strand as RNA or − strand as DNA.

Following the reactions the labeled components are purified using the Qiagen PCR Purification kit and concentrated.

The labeled products are then denatured at 95° C. for 5 minutes, diluted into Agilents Deposition Hybridization buffer and transferred to an Agilent Human 1 cDNA microarray. The array is allowed to hybridize overnight at 65° C., washed, scanned and featured extracted according to manufacturers instructions.

Transcripts present at higher concentrations in one sample are recognized as either having higher Cyanine 3 or Cyanine 5 signals.

The above results and discussion demonstrate that novel methods of producing linearly amplified amounts of (+) strand RNA from an initial mRNA source are provided. The subject methods provide for an important new tool for molecular biological applications, where it is desired to employ (+) strand RNA as opposed to antisense RNA. As such, the subject methods represent a significant contribution to the art.

All publications and patent application cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method for producing linearly amplified amounts of (+) strand RNA, said method comprising:
(a) producing double-stranded cDNA from an initial mRNA source by employing a second strand cDNA primer comprising an RNA polymerase promoter domain located at least proximal to its 5′ terminus; and
(b) transcribing said double-stranded cDNA into (+) strand RNA.

2. The method according to claim 1, wherein said second strand cDNA primer comprises an ATG codon located at least proximal to its 3′ terminus.

3. The method according to claim 2, wherein said second strand cDNA primer further comprises a spacer domain between said 5′ RNA polymerase promoter domain and said 3′ ATG codon.

4. The method according to claim 3, wherein said second strand cDNA primer is described by the formula:
5′-RNA polymerase promoter domain-(N)ₙ-ATG-(N)ₘ-3′
wherein:
N is any deoxyribonucleotide residue;
n is from 1 to 10; and
m is 0 or an integer from 1 to 10.

5. The method according to claim 1, wherein said producing step (a) comprises a first strand cDNA synthesis step and a second strand cDNA synthesis step.

6. The method according to claim 5, wherein a first polymerase is employed for synthesis of said first strand cDNA and a second polymerase is employed for synthesis of said second strand cDNA.

7. The method according to claim 1, wherein said double-stranded cDNA is separated from reverse transcriptase prior to said transcribing step (b).

8. The method according to claim 1, wherein said transcribing step (b) occurs in the presence of a reverse transcriptase that is incapable of RNA-dependent DNA polymerase activity during said transcribing step.

9. The method according to claim 1, wherein said initial mRNA source is total RNA.

10. The method according to claim 1, wherein said RNA polymerase promoter domain is chosen from a domain comprising the T7, Sp6 or T3 promoter.

11. A method for producing labeled deoxyribonucleic acid target molecules, said method comprising:
(a) producing (+) strand RNA from an initial mRNA source by a method comprising:
(i) producing double-stranded cDNA from said initial mRNA source by employing a second strand cDNA primer comprising an RNA polymerase promoter domain located at least proximal to its 5′ terminus; and
(i) transcribing said double-stranded cDNA into (+) strand antisense RNA to produce (+) strand mRNA; and
(b) employing said (+) strand mRNA as template to enzymatically produce said labeled deoxyribonucleic acid target molecules.

12. The method according to claim 11, wherein said second strand cDNA primer comprises an ATG codon located at least proximal to its 3′ terminus.

13. The method according to claim 12, wherein said second strand cDNA primer further comprises a spacer domain between said 5′ RNA polymerase promoter domain and said 3′ ATG codon.

14. The method according to claim 13, wherein said second strand cDNA primer is described by the formula:
5′-RNA polymerase promoter domain-(N)ₙ-ATG-(N)ₘ-3′
wherein:
N is any deoxyribonucleotide residue;
n is from 1 to 10; and
m is 0 or an integer from 1 to 10.

15. The method according to claim 11, wherein said producing step (a)(i) comprises a first strand cDNA synthesis step and a second strand cDNA synthesis step.

16. The method according to claim 15, wherein a first polymerase is employed for synthesis of a first portion of said first strand cDNA and a second polymerase is employed for synthesis of said second strand cDNA and a third polymerase is employed to complete said first strand synthesis.

17. The method according to claim 11, wherein said double-stranded cDNA is separated from reverse transcriptase prior to said transcribing step (a)(ii).

18. The method according to claim 11, wherein said transcribing step (a)(ii) occurs in the presence of a reverse transcriptase that is incapable of RNA-dependent DNA polymerase activity during said transcribing step.

19. The method according to claim 11, wherein said initial mRNA source is total RNA.

20. The method according to claim 11, wherein said RNA polymerase promoter domain is chosen from a domain comprising the T7, SP6, or the T3 promoter.

21. A kit for use in linearly amplifying an initial mRNA source into (+) strand RNA, said kit comprising:
a second strand cDNA primer comprising an RNA polymerase promoter domain at its 5′ terminus; and
instructions for practicing the method according to claim 1.

22. The kit according to claim 21, wherein said second strand cDNA primer comprises an ATG codon at its 3′ terminus.

23. The kit according to claim 22, wherein said second strand cDNA primer further comprises a spacer domain between said 5′ RNA polymerase promoter domain and said 3′ ATG codon.

24. The kit according to claim 23, wherein said second strand cDNA primer is described by the formula:
5′-RNA polymerase promoter domain-(N)ₙ-ATG-(N)ₘ-3′
wherein:
N is any deoxyribonucleotide residue;
n is from 1 to 10; and
m is 0 or an integer from 1 to 10.

25. A method of detecting the presence of a nucleic acid analyte in a sample, said method comprising:
(a) producing labeled deoxyribonucleic acid target molecules from said sample according to the method of claim 11;
(b) contacting said labeled deoxyribonucleic acid target molecules with a nucleic acid array;
(c) detecting any binding complexes on the surface of the said array to obtain binding complex data; and
(d) determining the presence of said nucleic acid analyte in said sample using said binding complex data.

26. The method according to claim 25, wherein said method further comprises a data transmission step in which a result from a reading of the array is transmitted from a first location to a second location.

27. A method according to claim 26, wherein said second location is a remote location.

28. A method comprising receiving data representing a result of a reading obtained by the method of claim 25.

29. A hybridization assay comprising the steps of:
(a) contacting at least one labeled target nucleic acid sample produced according to the method of claim 1 with a nucleic acid array to produce a hybridization pattern; and
(b) detecting said hybridization pattern.

30. A second strand cDNA primer described by the formula:
5′-RNA polymerase promoter domain-(N)ₙ-ATG-(N)ₘ-3′
wherein:
N is any deoxyribonucleotide residue;
n is from 1 to 10; and
m is 0 or an integer from 1 to 10.
